(19) United States
(12) Patent Application Publication
(10) Pub. No.: US 2020/0355768 A1
(43) Pub. Date: Nov. 12, 2020

(54) COIL SYSTEM WITH DIFFERENT CURRENTS DRIVEN THROUGH THE SHIELD AND PRIMARY COILS

(71) Applicants: Geron BINDSEIL, Toronto (CA); Will HANDLER, Toronto (CA); Chad HARRIS, Toronto (CA)

(72) Inventors: Geron BINDSEIL, Toronto (CA); Will HANDLER, Toronto (CA); Chad HARRIS, Toronto (CA)

(21) Appl. No.: 16/408,987

(22) Filed: May 10, 2019

(51) Int. Cl. G01R 33/385; G01R 33/421; A61B 5/055

(52) U.S. Cl. CPC G01R 33/385; A61B 5/055; G01R 33/421

(57) ABSTRACT

The present disclosure provides a system and method for generating a magnetic field within a magnetic resonance imaging (MRI) apparatus having an imaging region. The coil system comprises a primary coil for generating the magnetic field in the MRI apparatus and is positioned at a first distance from the imaging region. The primary coil is also configured to be driven at a first current. The coil system also includes a shield coil positioned at a second distance, that is larger than the first distance, from the imaging region. The shield coil is configured to be driven at a second current that is different in magnitude than the first current. The shield coil is thus adapted to reduce the magnetic field outside of the shield coil when the primary coil is driven at the first current and the shield coil is driven at the second current.

### FIELD

The present disclosure relates generally to magnetic resonance (MR) imaging and more specifically, to a coil system and method for improving performance of the coils.

### BACKGROUND

Magnetic resonance imaging (MRI) systems generally use different types of coils and coil systems. Gradient coils, for example, typically have a coaxial double cylinder shape which includes an inner primary coil for generating the gradient magnetic field in an interior region by means of current distribution over a cylindrical surface. The gradient coils also include an outer shield coil for effectively canceling out “leaking” magnetic field from the primary coil in an exterior region by means of current distribution over a cylindrical surface enclosing and surrounding the primary coil.

Generally, when such a shielded gradient coil is driven with current, the internal electrical connections are designed so that current flows through the shield layer windings (or shield coil) in a directional sense opposite to that flowing through the primary layer windings (or primary coil) in order to reduce the strength of the magnetic fields that extends outside the gradient coil. The shield coil thus “shields” the outside environment from the magnetic fields generated by the inner primary coil.

Gradient coils are also generally designed and built such that each axis (e.g. X, Y, and Z axes) is driven independently by a single gradient amplifier output so that at any given time during the gradient current waveform, the magnitude of current passing through the primary windings is the same as the current passing through the shield windings. The gradient coils thus typically have a single pair of electrical connections (+/−) to the amplifier for each axis. Multiple electrical connection points may exist, for example for the purpose of bridging primary and shield windings to each other; however, these connections are typically arranged such that the primary and shield windings are driven in series by the amplifier.

In order to design a gradient coil with a very high efficiency (e.g. high gradient strength per Ampere) it is advantageous to position the primary layers close to the imaging volume or region and to position the shield coils at as great a distance as possible from the primary layers. This arrangement helps to advance the development of low-cost MRI systems by reducing cooling and power requirements.

However, if the shield coil layer is positioned at a large distance radially from the primary coil layer, the amount of current density required to provide effective shielding may be substantially less in the shield layer than the current density required in the primary layer during imaging. Therefore, when approximating an ideal continuous current density distribution using a discrete number of windings in the shield layer, if the current through each winding is the same in both the primary and shield layers, fewer windings are typically used in the shield layers to compensate for the lesser required current density. This design, however, may result in so few discrete windings present in the shield layer that the shielding effectiveness may be reduced significantly as a result of the discretization when compared with the ideal continuous current density distribution.

In addition, in discretizing the shield windings, the force and torque balance of the gradient coil axis when energized in the magnetic field of the main magnet may be affected negatively when compared with the ideal continuous current density design due to the imperfect approximation of the design.

### SUMMARY

According to an example aspect, the present disclosure provides a coil system for generating a magnetic field within a magnetic resonance imaging (MRI) apparatus having an imaging region, the coil system comprising: a primary coil for generating the magnetic field in the MRI apparatus and positioned at a first distance from the imaging region, the primary coil configured to be driven at a first current; and a shield coil positioned at a second distance, that is larger than the first distance, from the imaging region, the shield coil configured to be driven at a second current that is different in magnitude than the first current; wherein the shield coil is adapted to reduce the magnetic field generated by the primary coil outside of the shield coil when the primary coil is driven at the first current and the shield coil is driven at the second current.

According to another example aspect, the present disclosure provides a method of operating a coil system with a primary coil and a shield coil for generating a magnetic field in a magnetic resonance imaging (MRI) apparatus, the MRI apparatus having an imaging region, the method comprising: driving the primary coil with a first current for generating the magnetic field in the MRI apparatus, the primary coil being positioned at a first distance from the imaging region; and driving the shield coil with a second current, the second current being different in magnitude from the first current, the shield coil positioned at a second distance, that is larger than the first distance, from the imaging region; wherein the shield coil is adapted to reduce the magnetic field generated by the primary coil outside the shied coil when the primary coil is driven at the first current and the shield coil is driven at the second current.

### BRIEF DESCRIPTION OF THE DRAWINGS

Example embodiments of the present disclosure are provided in the following description. Such description makes reference to the annexed drawings wherein:

FIG. 1 is a block diagram of a magnetic resonance imaging (MRI) system;

FIG. 2 is a typical conventional symmetric transverse gradient coil with a sparse shield turn density;

FIG. 3 is a symmetric transverse gradient coil, designed in accordance with example embodiments of the present disclosure, with an increased shield turn density compared to the design shown in FIG. 2;

FIG. 4 is a schematic of a gradient coil system with half of a transverse gradient coil in accordance with an example embodiment of the present disclosure;

FIG. 5 is a schematic of a gradient coil system with half of a transverse gradient coil in accordance with another example embodiment of the present disclosure;

FIG. 6 is plot of the relative magnetic field outside a certain distance from the shield of the gradient coil of FIG. 2 versus the relative magnetic field at the same locations outside the gradient coil of FIG. 3; and

FIG. 7 is a flow chart showing an example method for operating a coil system for generating a magnetic field in a magnetic resonance imaging (MRI) apparatus.

FIG. 8 is a flow chart showing another example method for operating a coil system for generating a magnetic field in a magnetic resonance imaging (MRI) apparatus.

### DESCRIPTION OF EXAMPLE EMBODIMENTS

Conventional magnetic resonance imaging (MRI) systems represent an imaging modality which is primarily used to construct pictures of magnetic resonance (MR) signals from protons such as hydrogen atoms in an object. In medical MRI, typical signals of interest are MR signals from water and fat, the major hydrogen-containing components of tissues.

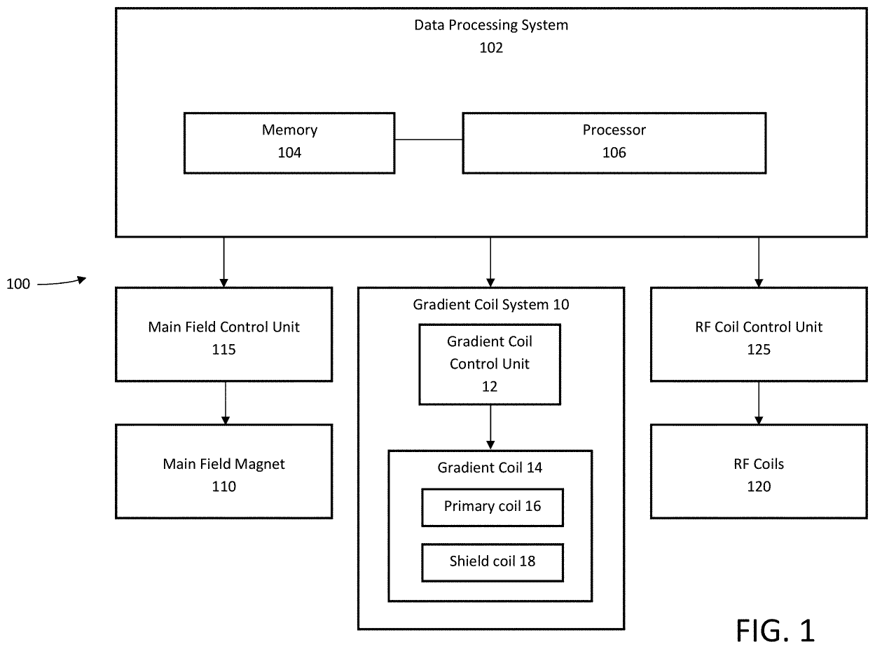

Referring to FIG. 1, a block diagram of an example magnetic resonance imaging (MRI) system is shown at 100 in accordance with an example embodiment. The example implementation of MRI system indicated at 100 is for illustrative purposes only, and variations including additional, fewer and/or varied components are possible.

As shown in FIG. 1, the illustrative MRI system 100 comprises a data processing system 102. The data processing system 102 can generally include one or more output devices such as a display, one or more input devices such as a keyboard and a mouse as well as one or more processors connected to a memory having volatile and persistent components. The data processing system 102 may further comprise one or more interfaces adapted for communication and data exchange with the hardware components of MRI system 100 used for performing a scan. As shown, data processing system 102 includes a memory 104 and a processor 106 for controlling the communication and data exchange with the hardware components.

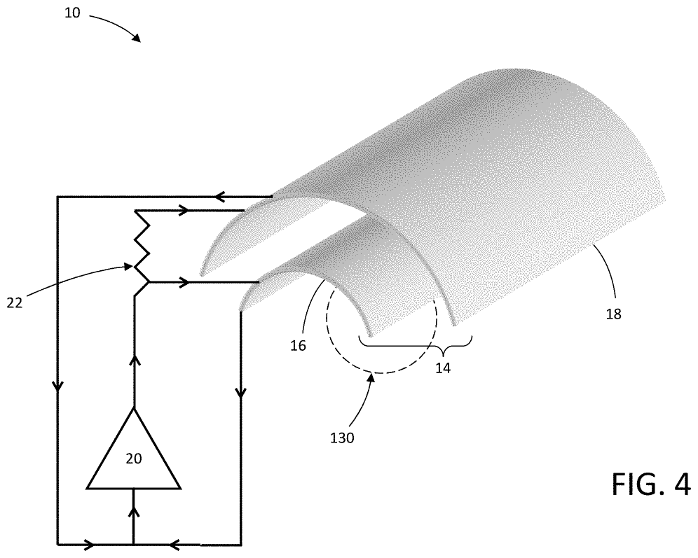

Continuing with FIG. 1, example MRI system 100 also includes a main field magnet 110. The main field magnet 110 may be implemented as a permanent, superconducting or a resistive magnet, for example. Other magnet types, including hybrid magnets suitable for use in MRI system 100 are contemplated. Main field magnet 110 is operable to produce a substantially uniform main magnetic field having strength B0 and a direction along an axis. The main magnetic field is used to create an imaging volume 130 (see FIGS. 3 and 4 for example) therein within which desired atomic nuclei, such as the protons in hydrogen within water and fat, of an object are magnetically aligned in preparation for a scan. In some implementations, as in this example implementation, a main field control unit 115 in communication with data processing system 105 may be used for controlling the operation of main field magnet 110.

The MRI system 100 further includes radio frequency (RF) coils 120. The RF coils 120 are used to establish an RF magnetic field with strength B1 to excite the atomic nuclei or “spins”. The RF coils 120 can also detect signals emitted from the “relaxing” spins within the object being imaged. Accordingly, the RF coils 120 may be in the form of separate transmit and receive coils or a combined transmit and receive coil with a switching mechanism for switching between transmit and receive modes.

The RF coils 120 may be implemented as surface coils, which are typically receive only coils and/or volume coils which can be receive and transmit coils. RF coils 120 can be integrated in the main field magnet 110 bore. Alternatively, RF coils 120 may be implemented in closer proximity to the object to be scanned, such as a head, and can take a shape that approximates the shape of the object, such as a close-fitting helmet. An RF coil control unit 125 in communication with data processing system 102 may be used to control the operation of the RF coils 120 in either a transmit aspect or a receive aspect.

To obtain images from the MRI system 100, one or more sets of RF pulses and gradient waveforms (collectively called “pulse sequences”) are selected at the data processing system 102. The data processing system 102 communicates the selected pulse sequence information to the RF control unit 125 and one or more gradient coil systems 10, discussed in more detail below, which collectively generate the associated waveforms and timings for providing a sequence of pulses to perform a scan.

The magnetic fields produced by gradient coil system 10, in combination and/or sequentially, can be superimposed on the main magnetic field such that selective spatial excitation of objects within gradient coils system 10 occur. In addition to allowing spatial excitation, gradient coil systems 10 may attach spatially specific frequency and phase information to the atomic nuclei placed within the imaging volume, allowing the resultant MR signal to be reconstructed into a useful image. A gradient coil control unit 12 in communication with data processing system 102 is used to control the operation of gradient coils 14. Generally, the imaging volume may be defined as the region in which MR images of interest are obtained using the MRI apparatus. The imaging volume may be a spherical volume 130 that is smaller than the total volume of space within gradient coil 14.

Gradient coil system 10 is generally used for encoding spatial information in the main magnetic field along at least one gradient axis, and typically encoding is performed along three perpendicular gradient axes. When there are three orthogonal gradient axes, the MRI system 100 may comprise three gradient coil systems 10, where each gradient coil system 10 may be configured to generate a magnetic field that varies along one of the three gradient axes.

As depicted, gradient coil system 10 includes gradient coil control unit 12 and one or more gradient coils 14. The size and configuration of the gradient coils 14 may be such that they produce a controlled and uniform linear gradient. For example, three paired orthogonal current-carrying gradient coils, along with the main field magnet 110, may be designed to produce a desired linear-gradient magnetic field along each axis.

The conductive components of gradient coil system 10, whether shielded or unshielded and including primary and shield coils, may include an electrical conductor (for example copper, aluminum, etc.). The internal electrical connections can be such that when a voltage difference is applied to the terminals of gradient coils 14, electric current can flow in the desired path. The conductive components for the three gradient axes for both primary coils 16 and shield coils 18 may be insulated by physical separation and/or a non-conductive barrier.

In examples of the present disclosure, each gradient coil 14 has a primary coil 16 and a shield coil 18, where primary coil 16 is shielded or surrounded by outer shield coil 18. Shield coil 18 can produce a magnetic field to counter the magnetic field produced by the inner primary gradient coil 16 in the region outside the shield coil. Thus, they collectively form a primary-shield coil pair. The shapes and sizes, conductor wire patterns and sizes, and current amplitudes and patterns of the primary-shield coil pairs can be selected so that the net magnetic field outside the gradient coils 14 is reduced (compared to where no shield coil 18 is used), for example, reduced to as close to zero as possible. For cylindrical magnets, for example, the two coils 16, 18 may be arranged in the form of concentric cylinders, whereas for vertical field magnets, the two coils 16, 18 may be arranged in coaxial, generally planar, disks.

In one embodiment, primary coil 16 has a first radius and shield coil 18 has a second radius that is greater than the first radius. In example implementations, such as when gradient coil system 10 is used to image a patient's head, the first radius may be in the range of about 15 cm to 30 cm, and the second radius may be in the range of about 20 cm to 50 cm (however the second radius is selected to be larger than the first radius). In other example implementations, such a coil system 10 may be used to image smaller subjects, such as small animals, infants and children. Shield coil 18 is positioned coaxially with, and radially disposed from, primary coil 16.

In such a coil pair, the “primary” coils can be responsible for creating the internal gradient field and the “shield” coils can be responsible for reducing the stray field of primary coil 16 outside the volume of space within the coil pair.

Each coil in a gradient coil for the X axis and Y axis may be configured as distributed windings in a “fingerprint” coil design. The gradient coil for the Z axis may consist of turns that resemble a set of solenoids. The windings may be formed by multiple thin metallic strips or large copper sheets on which are etched or cut winding patterns which are then formed into a cylinder or by wire wound into a cylindrical form. The wire can be multi-stranded, solid or hollow (e.g., to support the flow of coolant), among other possibilities.

Shield coils typically have a winding density that is designed to approximate a continuous current density distribution over the surface (e.g., cylindrical surface) of the shield coil when current runs therethrough, in order to cancel out the magnetic field generated by the primary coil, as noted above. Conventionally, the same current is driven through both the primary coil and the shield coil, where the current running through the shield coil is in a directional sense opposite to the current running through the primary coil.

As understood by the skilled person, since shield coil 18 is spaced apart from primary coil 16, the magnetic field to be generated by shield coil 18 to help cancel the magnetic field generated by the primary coil 16 should be weaker than the magnetic field to be generated by the primary coil 16. However, since the same current is conventionally driven through primary coil 16 and shield coil 18, a sparser winding density is conventionally used to achieve the desired weaker magnetic field.

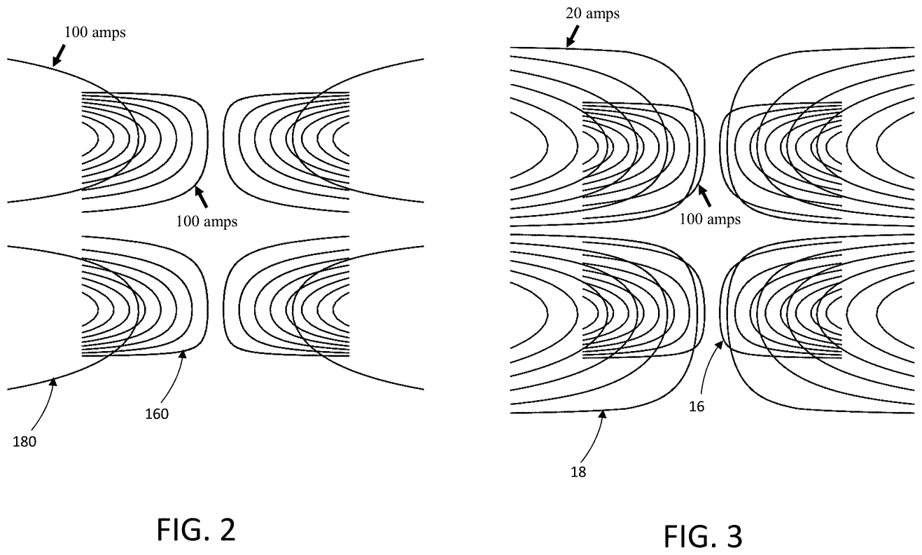

For example, as shown in FIG. 2, there is shown a conventional symmetric transverse gradient coil with a conventional shield coil 180 and a conventional primary coil 160. The shield turn density of the shield coil 180 is sparse (a single turn is depicted) when the conventional shield and primary coils 160, 180 are driven with the same current, for example 100A (i.e. amps).

In an example embodiment of the present disclosure, shown in FIG. 3, primary coil 16 is configured to be driven at a first current and shield coil 18 is configured to be driven at a second current that is different in magnitude than the first current. In other words, gradient coil system 10 is designed to drive different current magnitudes through primary coil 16 and shield coil 18.

When different currents are driven through primary coil 16 and shield coil 18, a closer approximation of the continuous current density distribution (as compared to that created in FIG. 2) may be achieved by changing the shield coil windings. For example, the number of windings on shield coil 18 may be increased by a factor that corresponds with a decrease in the second current, to better approximate the continuous current density distribution.

As depicted in FIG. 3, for example, having a factor of 5 increase in the number of shield windings allows for a corresponding factor of 5 reduction in the shield (or second) current as compared with the first current running through primary coil 16. FIG. 3 illustrates a current of 20A being driven through shield coil 18 while a current of 100A is driven through primary coil 16. Compared to the conventional design (e.g., as shown in FIG. 2), the increase factor in the number of shield windings may be enabled by the corresponding decrease factor in the current level for the shield coil.

In another example, at a given moment in time during a gradient waveform, primary coil 16 may be driven by 500A running through each primary wire turn, and shield coil 18 of the same axis may be driven by 50A running through each shield wire turn. In this manner, the winding density may be increased by a factor of 10 in shield coil 18 (compared to the conventional design), thereby better approximating the ideal designed current density distribution of the shield layer.

Shield coil 18, thus configured, may more effectively reduce the magnetic field outside gradient coil 14 when primary coil 16 is driven at the first current and shield coil 18 is driven at the second current. In that regard, the first current provided to primary coil 16 tends to be greater than the second current provided to shield coil 18.

Furthermore, the first current and the second current are also preferably linearly proportional. For example, during an imaging sequence, when the first current driven through the primary coil is 5A, the second current driven through the shield coil may be 2A. At a later point during the imaging sequence, if the first (primary) current is then −10A, the second (shield) current may be adjusted proportionally to −4A.

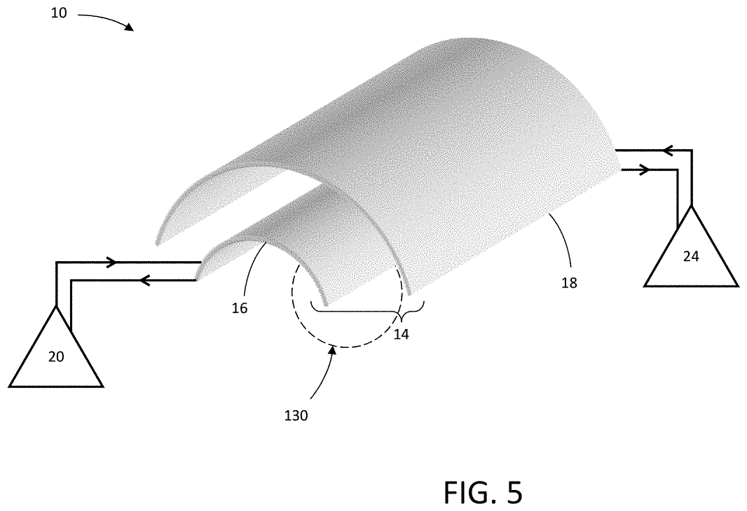

Driving primary coil 16 and shield coil 18 with different currents may be implemented in a number of ways. As depicted in FIGS. 4 and 5, gradient coil system 10 may include a first amplifier 20 and a current splitter 22 (e.g., a resistor or variable resistor). First amplifier 20 may be electrically coupled to both primary coil 16 and shield coil 18, providing the first current to primary coil 16. Current splitter 22 may be coupled to shield coil 18 for providing the second current to shield coil 18 at a reduced magnitude as compared to the first current from first amplifier 20. For example, the electrical characteristics of the current splitter may be adjusted to ensure the relative ratio of the first current to the second current is kept substantially constant over time.

In one implementation, as shown in FIG. 4, primary coil 16 and shield coil 18 are electrically coupled to first amplifier 20 in parallel and current splitter 22 is coupled between first amplifier 20 and both primary coil 16 and shield coil 18. The current flowing from first amplifier 20 thus flows through current splitter 22, which is configured to divide the current into the first current for primary coil 16, and into the second current for shield coil 18. The first current is then directed to primary coil 16 and the second current is directed to shield coil 18. The current from both primary coil 16 and shield coil 18 are merged back together before returning to first amplifier 20.

In another possible implementation, as shown in FIG. 5, gradient coil system 10 may include two independent amplifiers, first amplifier 20 and a second amplifier 24. As described above, first amplifier 20 may be coupled to primary coil 16 for providing the first current to primary coil 16. Second amplifier 24 may be electrically coupled to shield coil 18 for providing the second current to shield coil 18.

To maintain good fidelity of the gradient waveforms, first and second amplifiers 20, 24 may be configured to have sufficient voltage headroom and bandwidth to compensate for any opposing electromotive force induced by the mutual inductance coupling of primary coil 16 and shield coil 18. First and second amplifiers 20, 24 may be tuned to provide varying levels of current to primary coil 16 and shield coil 18 to ensure the relative ratio of the first current to the second current is kept substantially constant over time.

It is also possible to have more than two layers of coils for any given gradient axis that together form gradient coil 14. For example, primary coil 16 may have two coil layers, while shield coil 18 has a single coil layer. In such a case, the two inner coil layers of primary coil 16 may be driven with the first current and the outer coil layer of shield coil 18 may be driven with the second current.

For example, one or more primary layers may be designed first and then subsequently one or more shield layers may be designed. Alternately, all layers may be simultaneously designed where the final magnetic field is simply the sum of the magnetic fields generated by all the layers to produce a gradient field in the imaging volume and low field leaking outside the gradient. Gradient coil system 10 may reduce eddy currents and other interference which can cause artefacts in the scanned images. Since eddy currents mainly flow in conducting components of the MRI system 100 that are caused by magnetic fields outside of the gradient coils (fringe fields), reducing the fringe fields produced by gradient coils 14 may reduce interference.

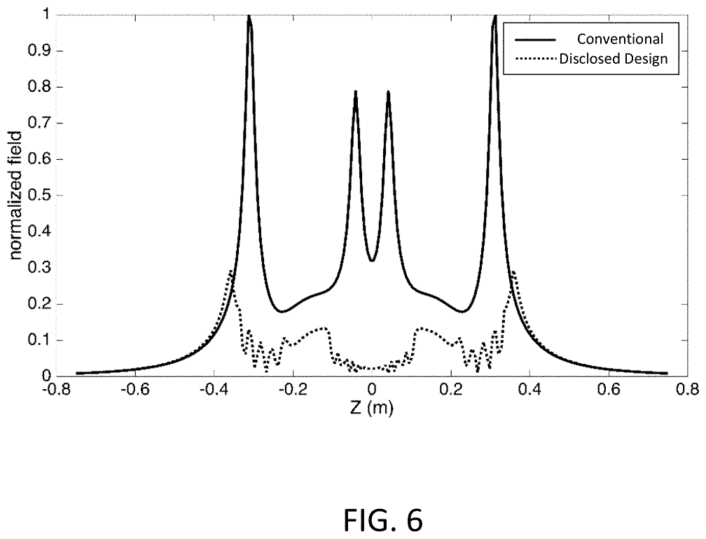

To compare the shielding effectiveness of the examples depicted in FIGS. 2 and 3, FIG. 6 illustrates a plot of relative magnetic field magnitude as a function of Z at a radial distance of 1 cm beyond the shield winding radius along the axis of the coil. The conventional gradient coil system of FIG. 2 is depicted in FIG. 6 as solid lines, while an example of the presently disclosed gradient coil system of FIG. 3 (with increased shield winding density) is depicted in dotted lines.

The maximum magnitude of magnetic field at a radial distance of 1 cm beyond the shield coil in both cases was computed on a cylindrical surface surrounding the coil design with the current set such that the two designs produced the same gradient strength at their centers. The maximum field outside the coil was found to be a factor of approximately 4 times worse for the conventional gradient coil represented in FIG. 2 as when compared with the example disclosed gradient coil 14 represented in FIG. 3. Both coils were found to have nearly identical efficiency and linearity. As readily understood by the skilled person, an improvement in external leakage field of approximately a factor of 4 is apparent.

In the above examples, while a cylindrical gradient coil system 10 has been described, other coil systems 10 for generating a magnetic field within an MRI apparatus are contemplated according to the present disclosure. In that manner, coil system 10 may involve a shielded shim coil or a field shifting coil within MRI apparatus 100, rather than a gradient coil.

In each case, coil systems 10 have a primary coil 16 for generating the magnetic field in MRI apparatus 100 at the first current. Primary coil 16 is also positioned at a first distance from imaging volume 130. Shield coil 18, in turn, is positioned at a second distance, that is larger than the first distance, from imaging volume 130. In other words, primary coil 16 is positioned closer to imaging region 130 than shield coil 18. In many applications, the configuration situates primary coil 16 between shield coil 18 and imaging region 130.

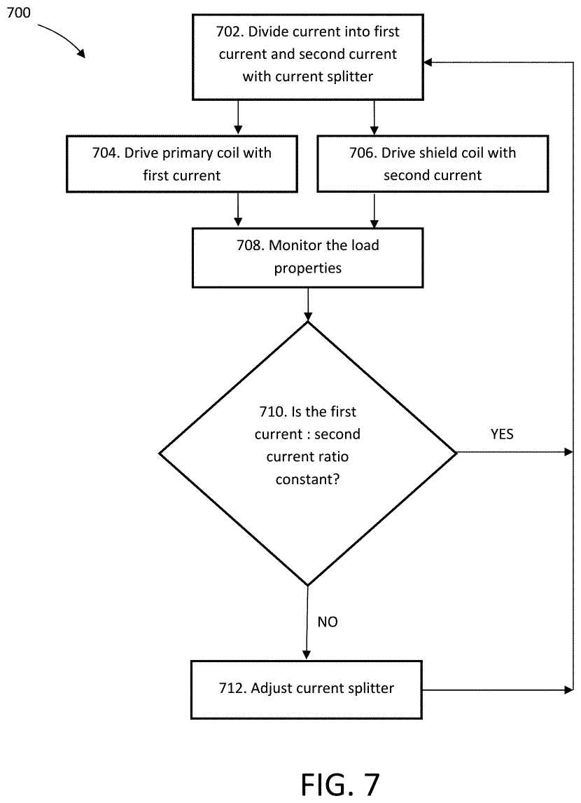

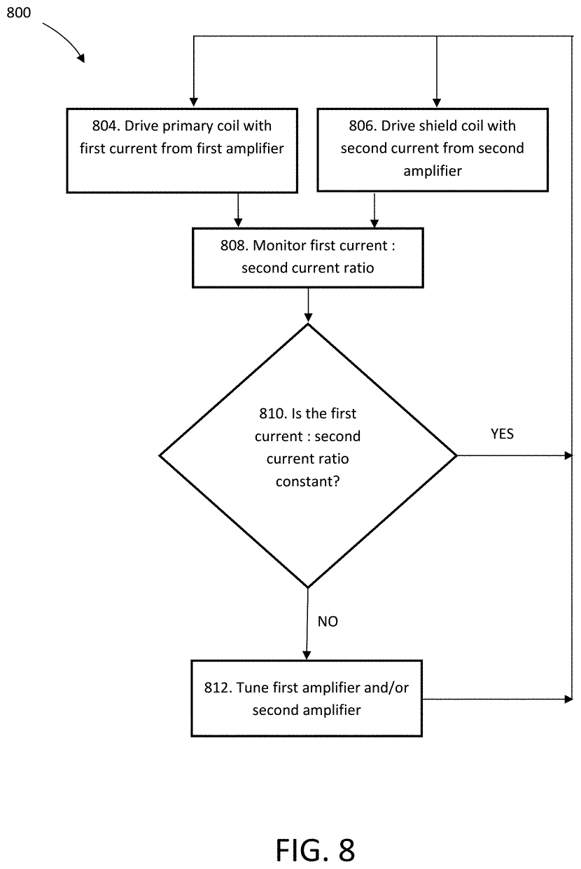

As discussed before, shield coil 18 is configured to be driven at the second current that is different in magnitude than the first current that is used to drive primary coil 16. Shield coil 18 is thus adapted to reduce the magnetic field generated by primary coil 16 outside of shield coil 18 when primary coil 16 is driven at the first current and shield coil 18 is driven at the second current. Referring now to FIGS. 7 and 8, methods of operating a coil system for generating a magnetic field in a magnetic resonance imaging (MRI) apparatus is indicated generally at 700 and 800. In some examples, methods 700 and 800 may be at least in part operated using the MRI system 100 as shown in FIG. 1 and, respectively, the coil system 10 shown in FIG. 4 (for FIG. 7) and the coil system 10 shown in FIG. 5 (for FIG. 8). Additionally, the following discussion of method 700 and 800 leads to further understanding of system 100. However, it is to be understood that system 100, and methods 700 and 800 can be varied, and need not work exactly as discussed herein in conjunction with each other, and that such variations are within scope of the appended claims.

To operate the coil system, as shown in FIG. 7, a primary coil is driven with a first current to produce a magnetic field within MRI apparatus 100 at 704. The primary coil is positioned at a first distance from the imaging region of the MRI apparatus. A shield coil is driven at 706 with a second current, where the second current is different in magnitude from the first current. The shield coil is also positioned at a second distance that is larger than the first distance, from the imaging region. In this manner, the primary coil is positioned closer to the imaging region than the shield coil. The shield coil thus reduces the magnetic field generated by the primary coil outside of the gradient coil system when the primary coil is driven at the first current and the shield coil is driven at the second current.

As presently described, the first current provided to the primary coil may be greater than the second current provided to the shield coil. The shield coil used may be designed so that the winding density is increased in order to better approximate the continuous current density distribution, as discussed above. The number of windings on the shield coil may be increased by a factor that corresponds with a decrease in the second current, to approximate the continuous current density distribution. As noted above, the increase factor in the number of shield windings and the corresponding decrease factor in the second current for the shield coil is relative to the typical case when the current running through the primary coil is the same as the current running through the shield coil.

In certain implementations, the initial current may be provided by a first amplifier and then divided at 702, such as using a current splitter (e.g., a resistor), into the first current and the second current before the currents are provided to the respective primary and shield coils.

During an imaging sequence, the shield coil may heat up in a way that changes its resistance in a way different than how the resistance of the primary coil changes. Other factors may also cause the load of the primary coil and shield coil to change relative to each other over time. The change in load over time may have the effect of changing the relative ratio of the first and second current directed through the primary and shield coil. This is undesirable because the winding density cannot be changed once the coils are built. It is the winding density that determines what the ratio between first current and second current need to be to approximate the ideal current density of the design.

As such, at 708, the first current and the second current are monitored to ensure the relative ratio of the first current to the second current is kept substantially constant over time. In cases where a first amplifier and a current splitter are used, the electrical characteristics of the current splitter may be adjusted to ensure the relative ratio of the first current to the second current is kept substantially constant over time. The current splitter may be a variable resistor or another device that divides current and can be controlled to adjust the ratio of the currents as required.

For example, to monitor the load properties, resistance may be monitored directly (e.g., using a direct measurement) or indirectly. The resistance may be monitored indirectly by measuring current in the primary coil current and the shield coil current lines (e.g., using any suitable current measuring devices, such as ammeters or current transducers). The resistance may also be monitored indirectly by monitoring the load properties of the primary and shield coils (such as resistance measurement through monitoring of voltage levels during use, for example). Resistance may also be monitored indirectly by monitoring temperatures of the primary and shield coils and inferring the resistance of the primary and shield (e.g., higher temperatures are indicative of higher resistance).

If it is determined at 710 that the first current and second current ratio is maintained substantially constant at the desired ratio, then the primary and shield coils may continue to be driven with current from the current splitter without requiring any adjustment. However, if it is determined at 710 that the first current and second current ratio is drifting away from or is not at the desired ratio, then the current splitter may be adjusted at 712 in order to return to the desired ratio.

For example, where the current splitter is a variable resistor, the variable resistor may be adjusted to split the current to the primary coil and the shield coil to maintain the relative current ratio. The electronic properties of the current splitter may be adjusted electronically, for example through use of a digital potentiometer. A system of monitoring the relative currents may be used by a control loop to automatically adjust the electronic properties of the current splitter in response to monitored data, for example signals from current transducers or resistance measurements, in order to maintain the relative current ratio.

FIG. 8 is a flowchart illustrating a method 800 that is applicable in cases where first and second amplifiers are used. As shown in FIG. 8, the primary coil is driven with the first current from the first amplifier at 804, and the shield coil is driven with the second current form the second amplifier at 806. The first current to second current ratio is monitored at 808 (e.g., using any suitable direct or indirect monitoring method, such as discussed above with respect to step 708).

In such applications, a gradient coil control unit coupled to both amplifiers may be used to determine at 810 if the current ratio is constant. If the current ratio is substantially constant at the desired ratio, the method 800 returns to drive the primary and shield coils at 804 and 806. If the current ratio is drifting away from or is not at the desired ratio, the amplifiers are tuned or directed (e.g., using the gradient coil control unit coupled to both amplifiers), at 812, to maintain the relative ratio of the first current and the second current over time. The amplifiers may be current-controlled amplifiers that include automatic adjustment of driving voltages to compensate for changes in load resistance and thus maintain the desired current ratio between the first current and the second current over time.

For example, in either the example of FIG. 7 or the example of FIG. 8, when the primary current is 5A and the shield current is 2A, if the primary current changes to −10A, the shield current would be adjusted to −4A, in order to maintain a primary to shield current ratio of 5:2.

In accordance with examples disclosed herein, removing the constraint that the same current be used for the primary coil and the shield coil windings may make possible the design and construction of higher efficiency shielded gradient coil designs, where the shield coils may be placed at a larger radial distance from the primary coils. Such a gradient coil system may also help to improve force and torque balancing of gradient coils because the shield winding pattern may better match the ideal current density distribution as designed.

At least some aspects disclosed may be embodied, at least in part, in software. That is, some disclosed techniques and methods may be carried out in a computer system or other data processing system in response to its processor, such as a microprocessor, executing sequences of instructions contained in a memory, such as ROM, volatile RAM, non-volatile memory, cache or a remote storage device.

A computer readable storage medium may be used to store software and data which when executed by a data processing system causes the system to perform various methods or techniques of the present disclosure. The executable software and data may be stored in various places including for example ROM, volatile RAM, non-volatile memory and/or cache. Portions of this software and/or data may be stored in any one of these storage devices.

Examples of computer-readable storage media may include, but are not limited to, recordable and non-recordable type media such as volatile and non-volatile memory devices, read only memory (ROM), random access memory (RAM), flash memory devices, floppy and other removable disks, magnetic disk storage media, optical storage media (e.g., compact discs (CDs), digital versatile disks (DVDs), etc.), among others. The instructions can be embodied in digital and analog communication links for electrical, optical, acoustical or other forms of propagated signals, such as carrier waves, infrared signals, digital signals, and the like. The storage medium may be the internet cloud, or a computer readable storage medium such as a disc.

Furthermore, at least some of the methods described herein may be capable of being distributed in a computer program product comprising a computer readable medium that bears computer usable instructions for execution by one or more processors, to perform aspects of the methods described. The medium may be provided in various forms such as, but not limited to, one or more diskettes, compact disks, tapes, chips, USB keys, external hard drives, wire-line transmissions, satellite transmissions, internet transmissions or downloads, magnetic and electronic storage media, digital and analog signals, and the like. The computer useable instructions may also be in various forms, including compiled and non-compiled code.

At least some of the elements of the systems described herein may be implemented by software, or a combination of software and hardware. Elements of the system that are implemented via software may be written in a high-level procedural language such as object oriented programming or a scripting language. Accordingly, the program code may be written in C, C++, J++, or any other suitable programming language and may comprise modules or classes, as is known to those skilled in object oriented programming. At least some of the elements of the system that are implemented via software may be written in assembly language, machine language or firmware as needed. In either case, the program code can be stored on storage media or on a computer readable medium that is readable by a general or special purpose programmable computing device having a processor, an operating system and the associated hardware and software that is necessary to implement the functionality of at least one of the embodiments described herein. The program code, when read by the computing device, configures the computing device to operate in a new, specific and predefined manner in order to perform at least one of the methods described herein.

While the teachings described herein are in conjunction with various embodiments for illustrative purposes, it is not intended that the teachings be limited to such embodiments. On the contrary, the teachings described and illustrated herein encompass various alternatives, modifications, and equivalents, without departing from the described embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

1. A coil system for generating a magnetic field within a magnetic resonance imaging (MRI) apparatus having an imaging region, the coil system comprising:
a primary coil for generating the magnetic field in the MRI apparatus, the primary coil positioned at a first distance from the imaging region and configured to be driven at a first current; and
a shield coil positioned at a second distance, that is larger than the first distance, from the imaging region, the shield coil configured to be driven at a second current that is different in magnitude than the first current;
wherein the shield coil is adapted to reduce the magnetic field generated by the primary coil outside of the shield coil when the primary coil is driven at the first current and the shield coil is driven at the second current.

2. The coil system of claim 1, wherein the first current provided to the primary coil is greater than the second current provided to the shield coil.

3. The coil system of claim 2, wherein the shield coil has a winding density that is designed to approximate a continuous current density distribution when the second current runs therethrough.

4. The coil system of claim 3, wherein the number of windings on the shield coil is increased by a factor that corresponds with a decrease in the second current, to approximate the continuous current density distribution.

5. The coil system of claim 4, wherein the primary coil and the shield coil are generally planar.

6. The coil system of claim 4, wherein the primary coil and the shield coil are cylindrical, the primary coil having a first radius, the shield coil having a second radius that is greater than the first radius, the shield coil positioned coaxially about the primary coil.

7. The coil system of claim 6, wherein at least one of the primary coil and the shield has two or more coil layers.

8. The coil system of claim 6, wherein the first radius is in the range of about 15 cm to 30 cm, and the second radius is in the range of about 20 cm to 50 cm, wherein the second radius is greater than the first radius.

9. The coil system of claim 4, wherein the primary coil and the shield coil collectively form a gradient coil.

10. The coil system of claim 4, wherein the primary coil and the shield coil collectively form a shielded shim coil.

11. The coil system of claim 1, further comprising:
an amplifier electrically coupled to the primary coil and the shield coil, for providing current to the primary coil and the shield coil, and
a current splitter coupled between the first amplifier and the coils, the current splitter configured to divide the current from the first amplifier into the first current for the primary coil and into the second current for the shield coil at a reduced magnitude as compared to the first current.

12. The coil system of claim 11, wherein the current splitter is a variable resistor configured to adjust division of the currents to maintain a relatively constant first current to second current ratio over an imaging time period

13. The coil system of claim 1, further comprising:
a first amplifier electrically coupled to the primary coil for providing the first current to the gradient coil; and
a second amplifier electrically coupled to the shield coil for providing the second current to the shield coil.

14. A magnetic resonance imaging (MRI) system comprising:
the coil system of claim 8 for generating a magnetic field along at least one gradient axis.

15. The MRI system of claim 14, wherein there are three orthogonal gradient axes, the MRI system comprising three instances of the coil system of claim 8, each coil system being configured to generate a respective magnetic field that varies along a respective one axis.

16. A method of operating a coil system with a primary coil and a shield coil for generating a magnetic field in a magnetic resonance imaging (MRI) apparatus, the MRI apparatus having an imaging region, the method comprising:
driving the primary coil with a first current for generating the magnetic field in the MRI apparatus, the primary coil being positioned at a first distance from the imaging region; and
driving the shield coil with a second current, the second current being different in magnitude from the first current, the shield coil positioned at a second distance, that is larger than the first distance, from the imaging region;
wherein the shield coil is adapted to reduce the magnetic field generated by the primary coil outside the shield coil when the primary coil is driven at the first current and the shield coil is driven at the second current.

17. The method of claim 16, wherein the first current provided to the primary coil is greater than the second current provided to the shield coil.

18. The method of claim 17, wherein current is provided by an amplifier the method further comprising
dividing the current from the first amplifier into the first current for the primary coil and into the second current for the shield coil; and

19. The method of claim 18, further comprising:
adjusting the current splitter to maintain a relatively constant first current to second current ratio over an imaging time period.

20. The method of claim 17, wherein the first current is provided by a first amplifier and the second current is provided by a second amplifier, the method further comprising:
tuning the first and/or second amplifiers to maintain a relatively constant first current to second current ratio over an imaging time period.
